(19) United States
(12) Patent Application Publication
(10) Pub. No.: US 2018/0140176 A1
(43) Pub. Date: May 24, 2018

(54) SINUPLASTY DEVICE

(71) Applicant: Pradeep K. Sinha, Atlanta, GA (US)

(72) Inventor: Pradeep K. Sinha, Atlanta, GA (US)

(21) Appl. No.: 15/820,923

(22) Filed: Nov. 22, 2017

Related U.S. Application Data
(60) Provisional application No. 62/426,043, filed on Nov. 23, 2016.

(51) Int. Cl. A61B 1/233; A61B 1/00; A61B 1/005; A61B 1/008; A61B 1/018; A61B 17/24

(52) U.S. Cl. CPC A61B 1/233; A61B 1/00135; A61B 1/00133; A61B 1/00165; A61B 1/00174; A61B 1/06; A61B 1/008; A61B 1/018; A61B 17/24; A61B 1/00105; A61B 1/00195; A61B 1/0055

(57) ABSTRACT

A sinuplasty device includes a fiber optic probe and a guide configured to guide the fiber optic probe. The guide housing is coaxial with the fiber optic probe and surrounds at least a portion of the fiber optic probe. The sinuplasty device also includes an introducer coaxial with the guide and with the fiber optic probe. The introducer is exterior to the guide and is configured to be movable along the guide between the lens of the fiber optic probe and a distal end of the fiber optic probe.

### REFERENCE TO RELATED APPLICATIONS

This application claims the benefit of U.S. Provisional Application No. 62/426,043, filed Nov. 23, 2016 and entitled Sinuplasty Device, the content of which is hereby incorporated by reference in its entirety.

### FIELD OF THE INVENTION

This application relates to medical devices, and more particularly, to minimally invasive devices, systems, and methods for treating sinusitis and other ear, nose, and throat conditions.

### BACKGROUND

The bones in the nose contain a series of cavities known as paranasal sinuses. Referring to FIGS. 1A and 1B, paranasal sinuses are air-filled spaces in a human's head 10 that surround the nasal cavity 12. The paranasal sinuses include maxillary sinuses 14, which are located under the eyes 16, frontal sinuses 18, which are located above the eyes 16, ethmoidal sinuses 20, which are located between the eyes 16, and sphenoidal sinuses 22, which are located behind the eyes 16. These sinuses are lined with mucous-producing epithelial tissue and ultimately opening into the nasal cavity 12. In normal conditions, mucous produced by the epithelial tissue slowly drains out of each sinus through an opening, which is called an ostium, and into the nasal cavity 12. However, these ostia sometimes can become blocked due to infection, allergies, air pollution, structural problems of the nose, or various other factors that inflame the tissue or otherwise block the passageways. As one example, sinusitis is a condition where the paranasal sinuses are inflamed or infected due to bacteria, viruses, fungi, allergies, or various combinations of factors. Blockage can be acute (resulting in episodes of pain) or chronic.

While it is desirable to treat these blocked passages, treatment of these sinuses is complicated because each sinus presents its own set of challenges for gaining access to the sinus. For example, a surgeon trying to gain access to the frontal sinus 18 must navigate a thin passageway that includes many bends and turns over a relatively long distance (from a medical perspective) before the frontal sinus 18 is reached. Moreover, because the frontal sinuses 18 are proximate to the eyes 16 and the brain, a misstep during navigation, such as excess force applied to a passageway wall, has the potential to result in great harm to the patient. As another example, the surgeon trying to gain access to the sphenoid sinus 22 must almost blindly navigate the passageway to the sphenoid sinus 22 due to the location within the head 10. Moreover, navigation to the sphenoid sinus 22 is further complicated because the sphenoid sinus 22 is near the carotid artery and the skull base of the brain. As such, any missteps during navigation, such as excess force applied to areas of the passageway causing puncture of the carotid artery, will result in great harm to the patient. As a further example, the surgeon trying to gain access to the maxillary sinus 14, must navigate a small and thin passageway that includes a 135 degree turn to access the maxillary sinus 14. Therefore, because each sinus presents its own set of challenges for gaining access, currently available individual sinuplasty devices are ineffective for treating all of the maxillary sinuses 14, the frontal sinuses 18, and the sphenoidal sinuses 22 as a single device.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain examples, a sinuplasty device includes a fiber optic probe; a guide, wherein the guide is coaxial with the fiber optic probe and surrounds at least a portion of the fiber optic probe; and an introducer, wherein the introducer is coaxial with the guide and with the fiber optic probe, exterior to the guide, and movable along the guide.

In some examples, the fiber optic probe includes a connecting end and a viewing end, wherein the viewing end includes a lens.

In various examples, the lens includes a wide angle lens.

The connecting end of the fiber optic probe, in various aspects, is connected to a visualization device.

In some examples, the visualization device includes an eye-piece.

In some aspects, the visualization device includes a monitor screen.

The fiber optic probe, in various examples, includes a light delivery system.

The fiber optic probe, in some examples, includes a flexible material.

In various examples, the fiber optic probe is movable through the guide.

In some aspects, the guide includes an insertion end, a connecting end distal from the insertion end, a main portion between the insertion end and the connecting end, and a tip portion between the main portion and the insertion end.

The tip portion, in some examples, is at an angle between about 0 degrees and about 135 degrees relative to the main portion.

The tip portion, in various aspects, is a removable component of the guide.

In some examples, the tip portion is fixed relative to the main portion.

In various examples, the tip portion is movable relative to the main portion.

In some aspects, wherein the tip portion is malleable.

The tip portion, in some aspects, includes at least one of a frontal sinus tip portion dimensioned to be positioned within a frontal sinus of a patient, a sphenoidal sinus tip portion dimensioned to be positioned within a sphenoidal sinus of a patient, and a maxillary sinus tip portion dimensioned to be positioned within a maxillary sinus of a patient.

In various examples, the introducer includes an introducing surface configured to connect to an item to be delivered by the sinuplasty device within a patient.

In some examples, the introducing surface includes at least one of a sinuplasty balloon, a stent, a drill, a vacuum tube, a knife, a fluid tube, and a spray nozzle connected to the introducing surface of the introducer.

The introducer, in some aspects, includes a fixed diameter.

In various examples, the introducer includes a changeable diameter.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1A is a view of the paranasal sinuses within a patient.

FIG. 1B is another view of the paranasal sinuses within the patient.

FIG. 2A is a side view of a sinuplasty device including a fiber optic probe, a guide, and an introducer according to an aspect of the current disclosure.

FIG. 2B is another side view of the sinuplasty device of FIG. 2A.

FIG. 2C is a side view of a sinuplasty device according to an aspect of the present disclosure.

FIG. 3 is a perspective view of a guide of a sinuplasty device according to an aspect of the current disclosure.

FIG. 4 is a detailed view of the sinuplasty device of FIG. 2A with an item on the introducer.

FIG. 5 is a sectional view of the sinuplasty device of FIG. 2A taken along line 5-5 in FIG. 2A.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a band” can include two or more such bands unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements, and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment. Directional references such as “up,” “down,” “top,” “left,” “right,” “front,” “back,” and “corners,” among others are intended to refer to the orientation as illustrated and described in the figure (or figures) to which the components and directions are referencing.

Various implementations described in the present disclosure can include additional systems, methods, features, and advantages, which can not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

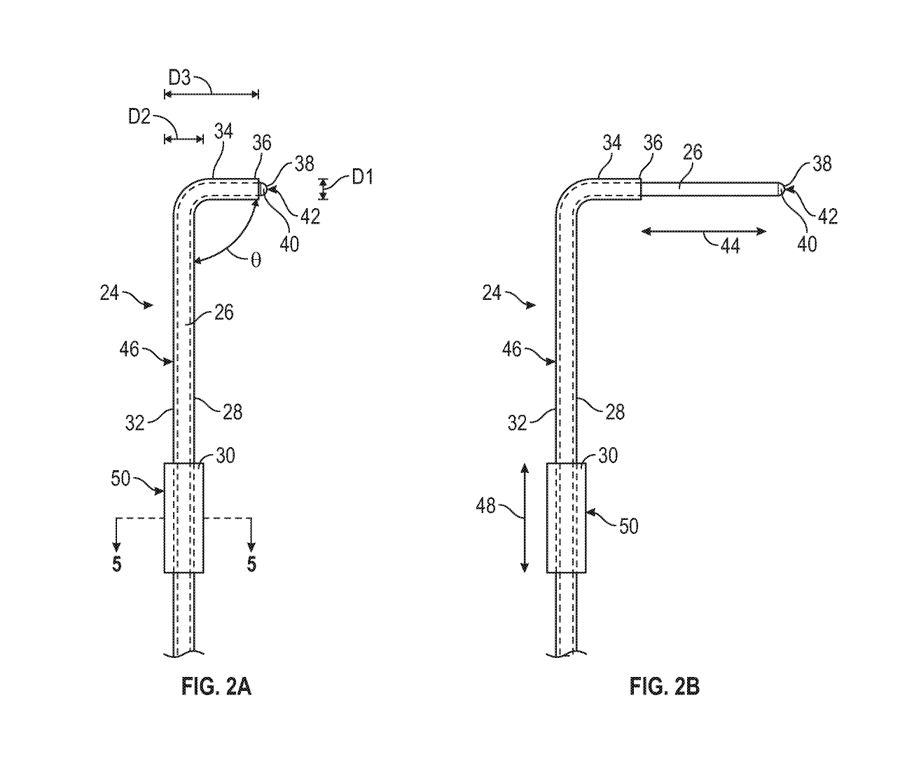

Referring to FIGS. 2A and 2B, in one aspect, disclosed is a sinuplasty device 24 and associated methods, systems, devices, and various apparatus. As described in detail below, the sinuplasty device 24 is a combination visualization and introduction device that can be used to gain access to the paranasal sinuses and introduce various items to the paranasal sinuses. It would be understood by one of skill in the art that the disclosed sinuplasty device 24 is described in but a few exemplary aspects among many.

In various aspects, the sinuplasty device 24 includes a fiber optic probe 26, a guide 28, and an introducer 30. The guide 28 is a housing having an insertion end 36 and a connecting end (not shown). In various aspects, the insertion end 36 is configured to be inserted into the human body while the connecting end is configured to either engage a holder that can be held by a person using the sinuplasty device 24 or serve as the holder that the person can directly hold. The guide 28 includes main portion 32 between the insertion end 36 and the connecting end, and a tip portion 34 between the main portion 32 and the insertion end 36. As described in detail below, the guide 28 is hollow such that the fiber optic probe 26 is movable through the guide 28.

As illustrated in FIG. 2A, the tip portion 34 is at an angle θ relative to the main portion 32. In some aspects, the angle θ is an angle suitable for gaining access to at least one of the maxillary sinus 14, the frontal sinus 18, or the sphenoidal sinuses 22. In these aspects, the angle θ can be between about 0° and about 135°. For example and without limitation, the angle can be 0°, 30°, 45°, 60°, 70°, 90°, 120°, or 135°.

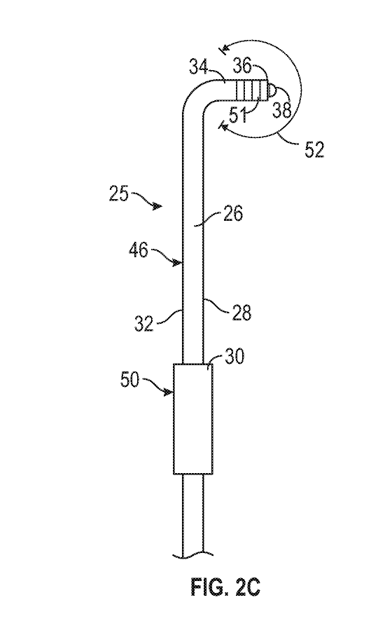

In some examples, the tip portion 34 is an articulating or malleable tip portion 34 that is movable to the various angles θ relative to the main portion 32. In this aspect, the angle θ of the tip portion 34 can be adjusted depending on which paranasal sinus is being accessed. Referring to FIG. 3, in other examples, the tip portions 34 are rigid such that the tip portions 34 are fixed at predefined angles θ, and each paranasal sinus may have a dedicated tip portion 34. The shape of each tip portion 34 may determine which tip portion 34 is associated with a particular sinus. For example and without limitation, the sinuplasty device 24 can include a removable fontal sinus tip portion, a removable sphenoidal sinus tip portion, and a removable maxillary sinus tip portion. Referring to FIG. 3, in one non-limiting example, the tip portion 34A is the removable sphenoidal sinus tip portion that is dedicated for use with the sinuplasty device 24 to gain access to the sphenoid sinus 22, and the tip portion 34B is the removable frontal sinus tip portion that is dedicated for use with the sinuplasty device 24 to gain access to the frontal sinus 18.

The tip portion 34 may also be malleable such that various shapes, designs, or configurations may be formed by the guide 28 as needed. For example and without limitation, in some cases, the tip portion 34 may be formed or shaped to comprise multiple angles.

In some cases, the tip portion 34 may be mechanically bendable or malleable. In these examples, the tip portions 34 may be an extension of a hand piece (not shown) for the guide 28 that the user may grasp. In various cases, the tip portion 34 is mechanically bendable or malleable through various mechanisms including, but not limited to gears, turn-wheels, motors, screws, and various other suitable movement mechanisms. These mechanisms may rotate the tip portion 34 through various angles and shapes to allow for coaxial approach to the target duct.

FIG. 2C illustrates another example of a sinuplasty device 25. The sinuplasty device 25 is substantially similar to the sinuplasty device 24 except that the tip portion 34 includes a number of articulating segments 51. Although four articulating segments 51 are illustrated, any number of articulating segments 51 may be provided including one articulating segment 51, two articulating segments 51, three articulating segments 51, or more than four articulating segments 51. In various examples, the articulating segments 51 are hingedly connected to adjacent articulating segments 51 and/or the probe 26 such that the tip portion 34 may articulate through an angle 52 in opposing directions. In various cases, the articulating segments 51 are constructed from various materials that are capable of bending while retaining some degree of rigidity similar, but not limited, to the materials of a bicycle chain, chainmail, or other similar. In one non-limiting example, the angle 52 is from 0° to about 270°, although various other ranges of the angle 52 may be provided. In some cases, the number of articulating segments 51 may allow for a narrower or wider angle 52 of articulation. As one non-limiting example, fewer articulating segments 51 may allow for a narrower angle 52 and additional segments 51 may allow for a greater angle 52. In some examples, the articulating segments 51 allow for articulation in at least one pair of opposing directions (e.g., articulation up and down or left to right). In other examples, the articulating segments 51 allow for articulating in two pairs of opposing directions (e.g., articulation both up and down and left to right). In various examples, the portion of the device 25 with the articulating segments 51 may be from about 7 mm to about 12 mm, although in other examples, the portion may be less than 7 mm or greater than 12 mm. In some examples, an internal opposing system, similar, but not limited, to a pulley system, would cause the tip portion 34 with articulating segments 51 to bend in opposing directions, such as up/down. In other cases, a second internal opposing system would cause the tip portion 34 and/or guide 28 to bend in perpendicular directions, such as left/right.

Referring back to FIGS. 2A-B, in examples where the tip portions 34 are rigid and at fixed angles θ, the tip portions 34 may be removably connected to the main portion 32 through threading, snap-fitting, or various other suitable mechanisms. In this manner, different tip portions 34 can be connected to and removed from the main portion 32 depending on which paranasal sinus is being accessed. In other examples where the tip portions 34 are at fixed angles θ, the main portion 32 and tip portion 34 are integrally formed as a single component guide 28. In these examples, multiple guides 28 can be utilized with the sinuplasty device 24, and each guide 28 can have the tip portion 34 at a different angle θ such that each guide 28 is dedicated to a different paranasal sinus. In this manner, the guide 28 used with the sinuplasty device 24 can be changed depending on which paranasal sinus is to be accessed.

In these examples where the tip portions 34 are removably connected to the main portion 32 or where different guides 28 dedicated to different paranasal sinuses can be interchangeably used with the sinuplasty device 24, the tip portions 34 and/or main portions 32 can be constructed from various autoclavable materials. For example and without limitation, the autoclavable materials may be various metals such as stainless steel or other comparable metals and alloys, various glass, plastics, or other composite materials, and various other suitable materials. In this aspect, the tip portions 34 can be reusable. In other aspects, the main portion 32 may also be constructed from various autoclavable materials such that the main portion 32 can be reusable. In various examples where the tip portions 34 are not removably connected to the main portion 32 and different shaped guides 28 are provided for each sinus, the entire guide 28 may be autoclavable.

The fiber optic probe 26 is the core of the sinuplasty device 24 and is configured to be moved through the guide 28. As described in detail below, the fiber optic probe 26 is coaxial with the guide 28, and the guide 28 surrounds at least a portion of the fiber optic probe 26. In various aspects, the fiber optic probe 26 includes a light delivery system (not shown) configured to illuminate those spaces into which the fiber optic probe 26 is inserted. The fiber optic probe 26 has a viewing end 38 and a connecting end (not illustrated). In various examples, the viewing end 38 includes a lens 40. The lens 40 is aspherical in various examples such that the lens 40 is configured to have a wide angle view. In these examples, a curved surface 42 of the aspherical lens 40 is configured to reduce the likelihood of traumatic impact by the fiber optic probe 26 as compared to a probe having a flat or angled surface. The connecting end of the fiber optic probe 26 is connected to a visualization output device, such as an eye-piece, monitors, or other suitable devices for outputting the view obtained through the viewing end 38.

The fiber optic probe 26 is flexible and has a thin diameter D1 such that the fiber optic probe 26 can access and navigate the various bends and turns of the passageways to each paranasal sinus. As a non-limiting example, the fiber optic probe 26 may be similar to that used with the product Marchal All-In-One Sialendoscope, sold by Karl Storz GmbH & Co. The Marchal All-In-One Sialendoscope may be similar to those fiber optic probes described in U.S. Pat. No. 9,351,530 or U.S. Pat. No. 7,850,604, the content of which is hereby incorporated by reference in their entireties. Various other suitable fiber optic probes 26 may be utilized. As illustrated in FIG. 2B by the arrow 44, the fiber optic probe 26 is movable through the guide 28 such that the viewing end 38 can be at a position proximate or distal to the insertion end 36 of the guide 28. In some aspects, the viewing end 38 can be movable to within an interior of the guide 28, although it need not be. In some examples, the fiber optic probe 26, the guide 28, or both may include a stopper to limit the extent to which the fiber optic probe 26 moves through the guide 28. This may provide a predetermined length at which the viewing end 38 can be positioned from the insertion end 36. In various cases, the predefined length may be from about 0 mm to about 150 mm. For example and without limitation, the predefined length may be from about 10 mm to about 140 mm, such as from about 20 mm to about 130 mm, such as from about 30 mm to about 120 mm, such as from about 40 mm to about 110 mm, such as from about 50 mm to about 100 mm, such as from about 50 mm to about 90 mm, such as from about 60 mm to about 80 mm. Various other suitable ranges for the predetermined length may be utilized.

The introducer 30 is coaxial with the guide 28 and the fiber optic probe 26 and configured to be movable along the guide 28, as indicated by the arrow 48 in FIG. 2B. In some examples, the introducer 30 is manually movable along the guide 28, such as through sliding or other suitable means. In other examples, the introducer 30 may be mechanically moved through various suitable mechanisms such as gears, turn-wheels, motors, screws, and various other suitable movement mechanisms. In various cases, the guide 28 may include a stopper to retain the introducer 30 on the guide 28. In this aspect, the introducer 30 is exterior to the guide 28 and is configured to move along at least a portion of an outer surface 46 of the guide 28 between the connecting end of the guide 28 and the insertion end 36 of the guide 28.

In some examples, the introducer 30 may also be movable along at least a portion of the probe 26 in addition to or in place of being movable along the guide 28. In some cases, the introducer 30 may be movable between the insertion end 36 of the guide and the viewing end 38 of the probe 26. In other cases, the introducer 30 may be movable between the connecting end of the guide 28 and the viewing end 38 of the probe 26, or between various other positions along the guide 28 and/or the probe 26. Similar to the guide 28, in cases where the introducer 30 is movable along the probe 26, the probe 26 may include a stopper that is configured to retain the introducer 30 on the sinuplasty device 24.

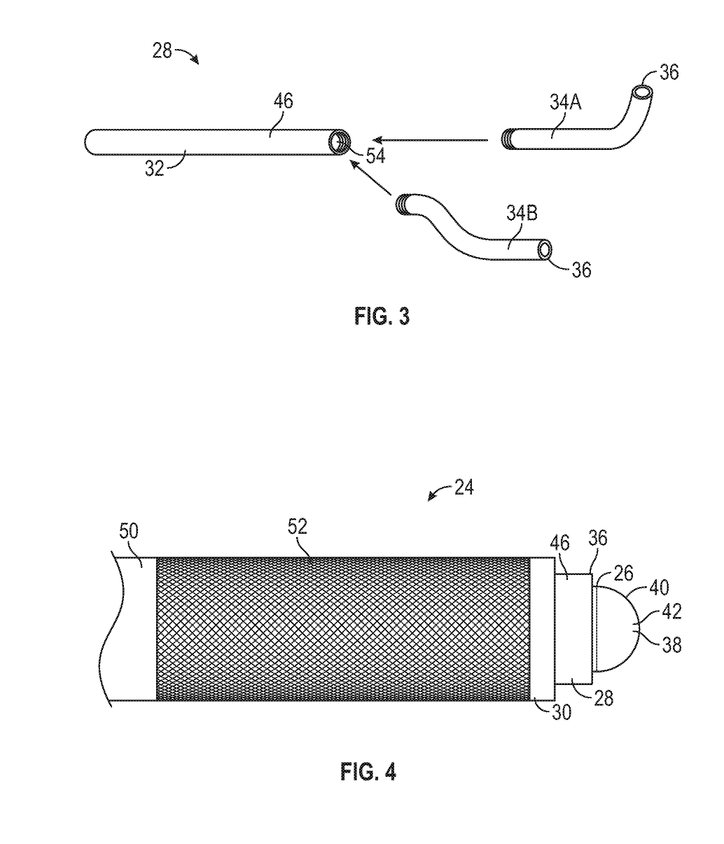

The introducer 30 defines an introducing surface 50 onto which a number of items to be introduced into the patient via the sinuplasty device 24 can be embedded, removably attached, or otherwise connected to the introducer 30 As one non-limiting example, FIG. 4 illustrates a stent 52 to be introduced into the patient that is removably attached to the introducer 30. In other examples, a balloon for dilating an opening within the patient, such as the ostium of one of the paranasal sinuses, may be attached. In other examples, the introducer 30 may define locations on the introducing surface 50 where various devices such as a drill, knife, vacuum tube, fluid tube or nozzle, spray tube or nozzle, other nozzles, or other devices to be used within the patient may be attached. In various other examples, the introducer 30 may define locations on the introducing surface 50 where various drugs to be released within the patient may be retained.

In various aspects, the introducer 30 may define a fixed diameter D2. However, in various other examples, the diameter of the introducer 30 is expandable such that the diameter of the introducer 30 can increase to the diameter D3 illustrated in FIG. 2A. In these examples, the diameter of the introducer 30 may be expandable through various expanding and contracting mechanisms including, but not limited to, rotating members, dilators, screws, hinges, pushable members, and various other suitable expanding and contracting mechanisms.

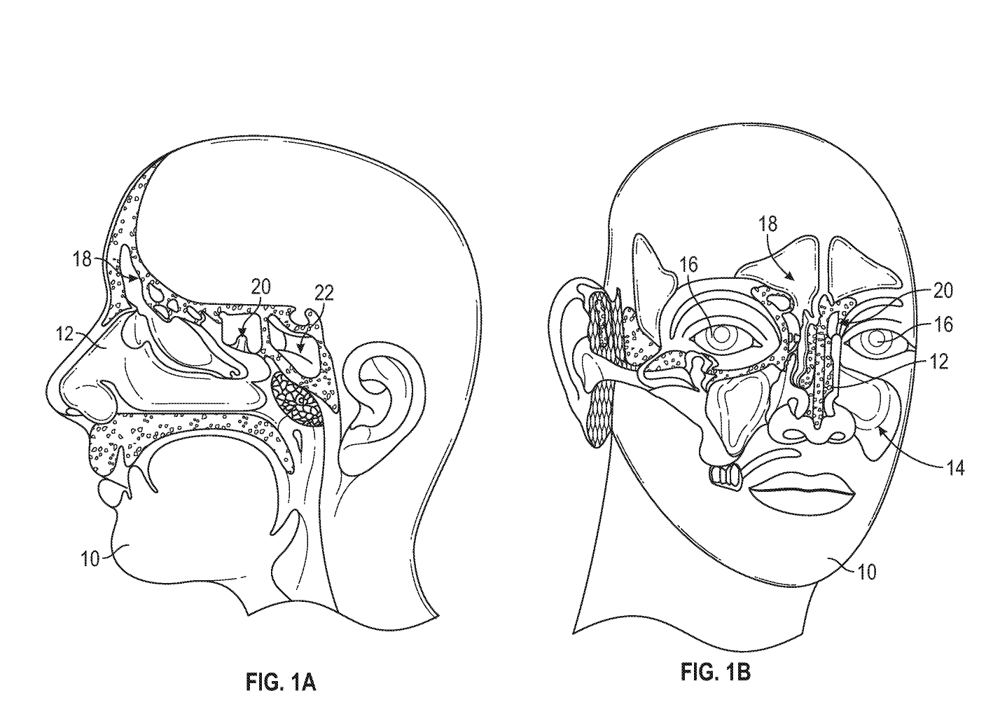

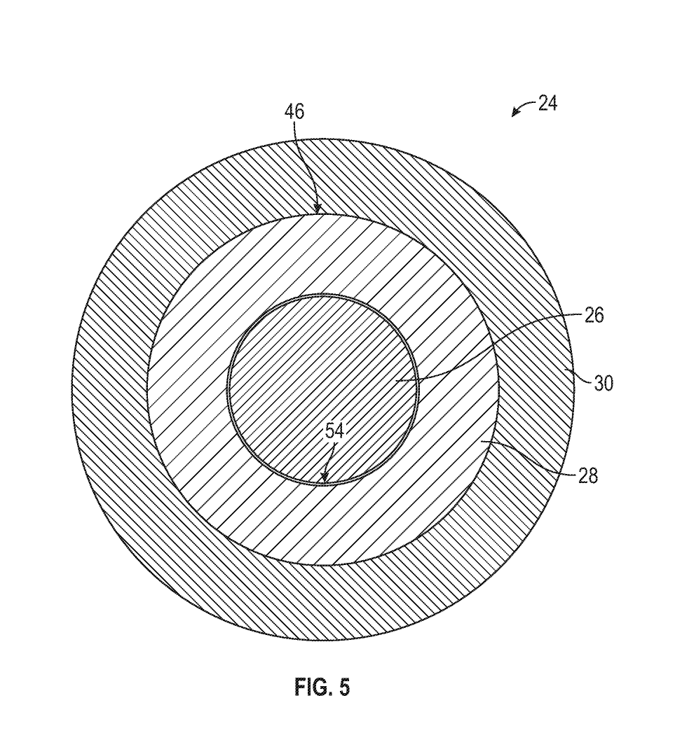

FIG. 5 illustrates a sectional view of the sinuplasty device 24 taken along line 5-5 in FIG. 1. As illustrated in FIG. 5, the fiber optic probe 26, guide 28, and introducer 30 are coaxial. The fiber optic probe 26 is movable within the interior of the guide 28 defined by an inner surface 54 of the guide 28. The introducer 30 is movable along the outer surface 46 of the guide 28.

In various examples, some or all of the components of the sinuplasty device 24 may be shaped and have various properties to aid the user using the sinuplasty device 24. For example, in some examples, at least a portion of the guide 28 may provide an ergonomic fit to the user's hand. In these examples, the portion of the guide 28 may be designed for grasping and may have tackiness to aid in grip, may have a shape for aiding in gripping, or may have various other properties or designs. As one non-limiting example, the portion of the guide 28 may have a triangular shape, although it need not.

A collection of exemplary embodiments, including at least some explicitly enumerated as “ECs” (Example Combinations), providing additional description of a variety of embodiment types in accordance with the concepts described herein are provided below. These examples are not meant to be mutually exclusive, exhaustive, or restrictive; and the invention is not limited to these example embodiments but rather encompasses all possible modifications and variations within the scope of the issued claims and their equivalents.

EC 1. A sinuplasty device comprising: a fiber optic cable core; a guide configured to guide the fiber optic cable core, the guide housing coaxial with the fiber optic cable core and surrounding at least a portion of the fiber optic cable core; and an introducer coaxial with the guide and with the fiber optic cable core, the introducer exterior to the guide and configured to be movable along the guide between the lens of the fiber optic cable core and a distal end of the fiber optic cable core.

EC 2. The device of any of the preceding or subsequent example combinations, wherein the fiber optic cable core comprises a viewing end, wherein the viewing end includes a lens.

EC 3. The device of any of the preceding or subsequent example combinations, wherein the connecting end of the fiber optic cable core is connected to a visualization device.

EC 4. The device of any of the preceding or subsequent example combinations, wherein the visualization device is an eye-piece.

EC 5. The device of any of the preceding or subsequent example combinations, wherein the visualization device includes a monitor screen.

EC. 6. The device of any of the preceding or subsequent example combinations, wherein the lens is a wide angle lens.

EC. 7 The device of any of the preceding or subsequent example combinations, wherein the fiber optic cable core further comprises a light delivery system.

EC 8. The device of any of the preceding or subsequent example combinations, wherein the fiber optic cable core is flexible.

EC 9. The device of any of the preceding or subsequent example combinations, wherein the guide comprises an insertion end and a connecting end distal from the insertion end, wherein the guide includes a main portion between the insertion end and the connecting end, and wherein the guide includes a tip portion between the main portion and the insertion end.

EC 10. The device of any of the preceding or subsequent example combinations, wherein the tip portion of the guide is at an angle between about 0 degrees and about 135 degrees relative to the main portion of the guide.

EC 11. The device of any of the preceding or subsequent example combinations, wherein the tip portion of the guide is a removable component of the guide.

EC 12. The device of any of the preceding or subsequent example combinations, wherein the tip portion of the guide is fixed relative to the main portion of the guide.

EC 13. The device of any of the preceding or subsequent example combinations, wherein the tip portion of the guide is movable relative to the main portion of the guide.

EC 14. The device of any of the preceding or subsequent example combinations, wherein the tip portion of the guide is a frontal sinus tip portion dimensioned to be positioned within a frontal sinus of a patient.

EC 15. The device of any of the preceding or subsequent example combinations, wherein the tip portion of the guide is a sphenoidal sinus tip portion dimensioned to be positioned within a sphenoidal sinus of a patient.

EC 16. The device of any of the preceding or subsequent example combinations, wherein the tip portion is a maxillary sinus tip portion dimensioned to be positioned within a maxillary sinus of a patient.

EC 17. The device of any of the preceding or subsequent example combinations, wherein the introducer defines an introducing surface configured to connect to an item to be delivered by the device within a patient.

EC 18. The device of any of the preceding or subsequent example combinations, further comprising a sinuplasty balloon connected to the introducing surface of the introducer.

EC 19. The device of any of the preceding or subsequent example combinations, further comprising a stent connected to the introducing surface of the introducer.

EC 20. The device of any of the preceding or subsequent example combinations, further comprising a drill connected to the introducing surface of the introducer.

EC 21. The device of any of the preceding or subsequent example combinations, further comprising a spray nozzle connected to the introducing surface of the introducer.

EC 22. The device of any of the preceding or subsequent example combinations, wherein a diameter of the introducer is fixed.

EC 23. The device of any of the preceding or subsequent example combinations, wherein a diameter of the introducer is configured to expand and contract.

It should be emphasized that the above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described invention, nor the claims that follow.

That which is claimed:

1. A sinuplasty device comprising:
a fiber optic probe;
a guide, wherein the guide is coaxial with the fiber optic probe and surrounds at least a portion of the fiber optic probe; and
an introducer, wherein the introducer is coaxial with the guide and with the fiber optic probe, exterior to the guide, and movable along the guide.

2. The sinuplasty device of claim 1, wherein the fiber optic probe comprises a connecting end and a viewing end, and wherein the viewing end includes a lens.

3. The sinuplasty device of claim 2, wherein the lens comprises a wide angle lens.

4. The sinuplasty device of claim 2, wherein the connecting end of the fiber optic probe is connected to a visualization device.

5. The sinuplasty device of claim 4, wherein the visualization device comprises an eye-piece.

6. The sinuplasty device of claim 4, wherein the visualization device comprises a monitor screen.

7. The sinuplasty device of claim 1, wherein the fiber optic probe comprises a light delivery system.

8. The sinuplasty device of claim 1, wherein the fiber optic probe comprises a flexible material.

9. The sinuplasty device of claim 1, wherein the fiber optic probe is movable through the guide.

10. The sinuplasty device of claim 1, wherein the guide comprises an insertion end, a connecting end distal from the insertion end, a main portion between the insertion end and the connecting end, and a tip portion between the main portion and the insertion end.

11. The sinuplasty device of claim 10, wherein the tip portion is at an angle between about 0 degrees and about 135 degrees relative to the main portion.

12. The sinuplasty device of claim 10, wherein the tip portion is a removable component of the guide.

13. The sinuplasty device of claim 10, wherein the tip portion is fixed relative to the main portion.

14. The sinuplasty device of claim 10, wherein the tip portion is movable relative to the main portion.

15. The sinuplasty device of claim 10, wherein the tip portion is malleable.

16. The sinuplasty device of claim 10, wherein the tip portion comprises at least one of a frontal sinus tip portion dimensioned to be positioned within a frontal sinus of a patient, a sphenoidal sinus tip portion dimensioned to be positioned within a sphenoidal sinus of a patient, and a maxillary sinus tip portion dimensioned to be positioned within a maxillary sinus of a patient.

17. The sinuplasty device of claim 1, wherein the introducer comprises an introducing surface configured to connect to an item to be delivered by the sinuplasty device within a patient.

18. The sinuplasty device of claim 17, further comprising at least one of a sinuplasty balloon, a stent, a drill, a vacuum tube, a knife, a fluid tube, and a spray nozzle connected to the introducing surface of the introducer.

19. The sinuplasty device of claim 1, wherein the introducer comprises a fixed diameter.

20. The sinuplasty device of claim 1, wherein the introducer comprises a changeable diameter.
